(19) United States
(12) Patent Application Publication
(10) Pub. No.: US 2026/0147005 A1
(43) Pub. Date: May 28, 2026

(54) BIOMARKER FOR DIAGNOSIS, PROGNOSIS, ASSESSMENT AND THERAPY STRATIFICATION

(71) Applicant: The University of Melbourne, Victoria (AU)

(72) Inventors: Laura Elizabeth Downie, Victoria (AU); Holly Rose Chinnery, Victoria (AU); Alexis Ceecee Britten-Jones, Victoria (AU); Mengliang Wu, Victoria (AU); Haihan Jiao, Victoria (AU)

(21) Appl. No.: 19/120,614

(22) PCT Filed: Oct. 12, 2023
(86) PCT No.: PCT/AU2023/051011

§ 371 Date: Apr. 11, 2025

(30) Foreign Application Priority Data
Oct. 12, 2022 (AU) 2022902982

(51) Int. Cl. G01N 33/74

(52) U.S. Cl. CPC G01N 33/74; G01N 2333/5755; G01N 2800/16

(57) ABSTRACT

The present disclosure relates to use of tear fluid neuropeptide Y (NPY) level for diagnosis, prognosis, assessment and therapy stratification of corneal nerve damage or loss, corneal neuropathy, corneal neuropathic pain, corneal neuralgia, ocular disease, or peripheral neuropathy.

### TECHNICAL FIELD

The present disclosure relates to clinical diagnosis. In particular the disclosure relates to the diagnosis and/or prognosis and/or therapy stratification of corneal nerve damage or loss, corneal neuropathy, corneal neuropathic pain, corneal neuralgia, ocular disease, or peripheral neuropathy.

### BACKGROUND OF INVENTION

Peripheral neuropathy, a result of damage to the nerves located outside of the brain and spinal cord (peripheral nerves), often causes weakness, numbness and pain, usually in the hands and feet. It can also affect other areas and body functions including digestion, urination and circulation.

The peripheral nervous system is made up of two divisions: the somatic nervous system and the autonomic nervous system. The somatic system contains sensory and motor neurons. It sends and receives sensory information and motor signals. The autonomic system is responsible for regulating involuntary body functions.

Peripheral neuropathy results when nerve cells, called neurons, are damaged or destroyed. This disrupts the way the neurons communicate with each other and with the brain. Neuropathy can affect one nerve (mononeuropathy) or nerve type, a combination of nerves in a limited area (multifocal neuropathy) or many peripheral nerves throughout the body (polyneuropathy).

Peripheral neuropathy can result from traumatic injuries, infections, metabolic problems, inherited causes and exposure to toxins. One of the most common causes is diabetes.

Diabetic peripheral neuropathy is one of the most common and severe microvascular complications of diabetes. As a major contributor to peripheral nerve injury, diabetic peripheral neuropathy impairs quality of life and potentiates risks of disability such as foot ulceration and amputation in diabetic subjects. When diabetic peripheral neuropathy is not detected early, disease progression is irreversible.

Thus, biomarkers for early diagnosis of peripheral neuropathy including diabetic peripheral neuropathy are needed.

### SUMMARY OF INVENTION

The present inventors have surprisingly found that the level of that neuropeptide Y (NPY) in tears can be used as an early-stage diagnostic marker of peripheral neuropathy (for example, corneal neuropathy, an ocular manifestation of peripheral neuropathy in, for example, individuals with diabetes) and can also be used to monitor changes to disease state further to therapeutic intervention.

Surprisingly, decreased levels of NPY in tears was found to be a marker of nerve damage or loss.

In addition to use in identification of peripheral neuropathy, stratification and for objective monitoring of treatment efficacy in patients, the identification of tear NPY as a new biomarker of neuropathy has an important role in the discovery and development of new treatments of early stage disease.

Accordingly, the present disclosure provides a method of diagnosing the presence of or pre-disposition for corneal nerve damage or loss in a subject, said method comprising the steps of:
a) determining the level of neuropeptide Y (NPY) in a tear fluid sample from the subject; and
b) comparing the level of the NPY to a reference level of the NPY,
wherein a decreased level of the NPY compared to the reference level is indicative of the presence of or pre-disposition for corneal nerve damage or loss.

In one embodiment, the decreased level of the NPY indicates reduced corneal nerve fibre length (CNFL) and/or corneal nerve fibre density (CNFD) in the subject.

The present disclosure also provides a method of diagnosing the presence of or pre-disposition for corneal neuropathy, corneal neuropathic pain, or corneal neuralgia, said method comprising the steps of:
a) determining the level of neuropeptide Y (NPY) in a tear fluid sample from the subject; and
b) comparing the level of the NPY to a reference level of the NPY,
wherein a decreased level of the NPY compared to the reference level indicates that indicates that said subject is suffering from or is at risk of corneal neuropathy, corneal neuropathic pain, or corneal neuralgia.

In one embodiment, the decreased level of the NPY indicates increased corneal nerve damage in the subject.

In one or a further embodiment, the decreased level of the NPY indicates reduced corneal nerve fibre length (CNFL) and/or corneal nerve fibre density (CNFD) in the subject.

The present disclosure also provides a method of diagnosing the presence of or pre-disposition for an ocular disease, said method comprising the steps of:
a) determining the level of neuropeptide Y (NPY) in a tear fluid sample from the subject; and
b) comparing the level of the NPY to a reference level of the NPY,
wherein a decreased level of the NPY compared to the reference level indicates that indicates that said subject is suffering from or is at risk of developing an ocular disease.

In one embodiment, the decreased level of the NPY indicates increased corneal nerve damage or loss in the subject.

In one or a further embodiment, the decreased level of the NPY indicates reduced corneal nerve fibre length (CNFL) and/or corneal nerve fibre density (CNFD) in the subject.

In some embodiments, the ocular disease is not dry eye disease.

The present disclosure also provides a method of diagnosing the presence of or pre-disposition for peripheral neuropathy, said method comprising the steps of:
a) determining the level of neuropeptide Y (NPY) in a tear fluid sample from the subject; and
b) comparing the level of the NPY to a reference level of the NPY,
wherein a decreased level of the NPY compared to the reference level indicates that indicates that said subject is suffering from or is at risk of developing peripheral neuropathy.

In one embodiment, the decreased level of the NPY indicates increased corneal nerve damage or loss in the subject.

In one or a further embodiment, the peripheral neuropathy is diabetic peripheral neuropathy.

In some embodiments, the decreased level of the NPY indicates reduced corneal nerve fibre length (CNFL) and/or corneal nerve fibre density (CNFD) in the subject.

In some embodiments, the decreased level of the NPY indicates reduced sural sensory nerve amplitude.

The present disclosure also provides a method of evaluating the level of corneal nerve damage or loss in a subject, said method comprising the steps of:
a) determining the level of neuropeptide Y (NPY) in a tear fluid sample from the subject
b) correlating the level of the NPY with severity of corneal nerve damage or loss.

The present disclosure also provides a method of evaluating the level of corneal neuropathy, corneal neuropathic pain, or corneal neuralgia, said method comprising the steps of:
a) determining the level of neuropeptide Y (NPY) in a tear fluid sample from the subject; and
b) correlating the level of the NPY with severity of corneal neuropathy, corneal neuropathic pain, or corneal neuralgia.

In one embodiment, the level of the NPY indicates the level of corneal nerve damage or loss, wherein a decreased level of NPY indicates increased corneal nerve damage or loss.

The present disclosure also provides a method of evaluating the level of peripheral neuropathy, said method comprising the steps of:
a) determining the level of neuropeptide Y (NPY) in a tear fluid sample from the subject; and
b) correlating the level of the NPY with severity of peripheral neuropathy.

In one embodiment, the level of the NPY indicates the level of corneal nerve damage or loss, wherein a decreased level of NPY indicates increased corneal nerve damage or loss.

In one or a further embodiment, the peripheral neuropathy is diabetic peripheral neuropathy.

In some embodiments, the method is for risk stratification or treatment stratification.

The present disclosure also provides a method of prognosis of a subject having corneal nerve damage or loss, the method comprising:
a) determining the level of neuropeptide Y (NPY) in a tear fluid sample from the subject; and
b) correlating said level of NPY to likely outcome of said corneal nerve damage or loss.

The present disclosure also provides a method of prognosis of a subject having corneal neuropathy, corneal neuropathic pain, corneal neuralgia, the method comprising:
a) determining the level of neuropeptide Y (NPY) in a tear fluid sample from the subject; and
b) correlating said level of NPY to likely outcome of said corneal neuropathy, corneal neuropathic pain, or corneal neuralgia.

In one embodiment, the NPY correlates with the level of corneal nerve damage or loss, wherein a decreased level of NPY indicates increased corneal nerve damage or loss.

The present disclosure also provides a method of prognosis of a subject having peripheral neuropathy, the method comprising:
a) determining the level of neuropeptide Y (NPY) in a tear fluid sample from the subject; and
b) correlating said level of NPY to likely outcome of said peripheral neuropathy.

In one embodiment, the level of the NPY correlates with the level of corneal nerve damage or loss, wherein a decreased level of NPY indicates increased corneal nerve damage or loss.

In one or a further embodiment, the peripheral neuropathy is diabetic peripheral neuropathy.

In some embodiments, the method is for monitoring treatment efficacy or disease progression.

The present disclosure also provides a method of monitoring treatment efficacy of a subject having corneal nerve damage or loss, corneal neuropathy, corneal neuropathic pain, corneal neuralgia, ocular disease, or peripheral neuropathy, the method comprising:
a) determining the level of neuropeptide Y (NPY) in a tear fluid sample from the subject; and
b) correlating said level of NPY to a reference level indicative of a responder or non-responder to the treatment, or comparing said level to a baseline level of NPY before initiation of treatment; and
optionally, continuing treatment when the subject is determined to be a responder or has an increased level of NPY compared to the baseline level; or switching the treatment when the subject is determined to be a non-responder, wherein switching the treatment comprises increasing a dose of the treatment administered, adding one or more of agents to the treatment regimen, discontinuing the treatment and initiating a different treatment regimen.

In one embodiment, the peripheral neuropathy is diabetic peripheral neuropathy.

In some embodiments, the methods of the disclosure, the NPY comprises an amino acid sequence shown in SEQ ID NO: 2, or a fragment thereof.

In some embodiments, the methods of the disclosure, the reference level of the NPY in a sample from a normal subject.

In some embodiments, the methods of the disclosure, the reference level of the NPY is a threshold level. The threshold level may be a cut-off value.

In some examples, the subject is diagnosed with having corneal nerve damage or loss, corneal neuropathy, corneal neuropathic pain, corneal neuralgia, ocular disease, or peripheral neuropathy when said determined NPY level is lower than about 12.5 ng/mL.

In some embodiments, the methods of the disclosure further comprise obtaining the tear fluid sample.

In some embodiments, the methods of the disclosure further comprise a psychological, behavioural, physiological and/or genetic assessment of the subject.

In some embodiments, the methods of the disclosure further comprise selecting a treatment or modifying a treatment, based on the diagnosis, evaluation, or prognosis.

In some embodiments, the methods of the disclosure further comprise treating a subject identified as having corneal nerve damage or loss or a predisposition thereto.

In other of further embodiments, the methods of the disclosure further comprise treating a subject identified as suffering from or is at risk of corneal neuropathy, corneal neuropathic pain, or corneal neuralgia.

In other or further embodiments, the methods of the disclosure further comprise treating a subject identified as suffering from or being at risk of developing peripheral neuropathy (for example, corneal neuropathy, an ocular manifestation of peripheral neuropathy in, for example, individuals with diabetes (e.g., diabetes mellitus).

The present disclosure also provides use of ocular tear fluid neuropeptide Y (NPY) as a biomarker for corneal nerve damage or loss, corneal neuropathy, corneal neuropathic pain, corneal neuralgia, ocular disease, or peripheral neuropathy.

The present disclosure is not to be limited in scope by the specific examples described herein, which are intended for the purpose of exemplification only. Functionally-equivalent products, compositions and methods are clearly within the scope of the present disclosure.

Any example/embodiment of the present disclosure herein shall be taken to apply mutatis mutandis to any other example/embodiment of the disclosure unless specifically stated otherwise.

### BRIEF DESCRIPTION OF DRAWINGS

FIG. 1. Comparison of tear NPY levels in individuals with diabetes vs healthy control participants without peripheral neuropathy symptoms. NPY levels were lower in the diabetes group compared with control group (mean±SD: diabetes: 12.50±0.64 vs control: 14.72±0.67 ng/mL; p=0.02).

FIG. 2. Comparison of tear NPY levels in individuals with a high likelihood of neuropathy (CNFD≤16.7 nerves/mm²) vs individuals with a low likelihood of neuropathy (CNFD>16.7 nerves/mm²). Tear NPY levels were lower in those with, versus without, a high likelihood of neuropathy (mean±SD: 10.78±0.81 ng/mL vs 14.07±0.58 ng/mL; p=0.0021).

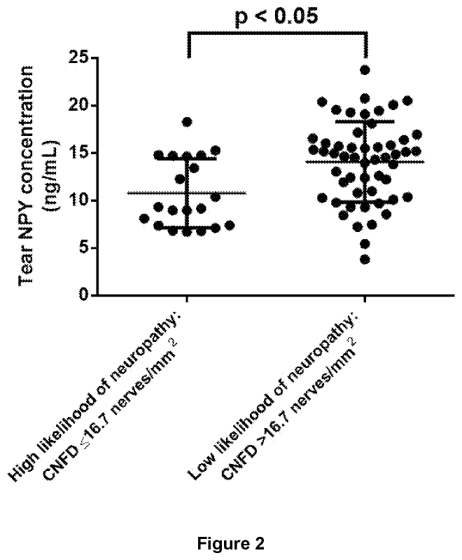

FIG. 3. A. Correlation between tear NPY level and central corneal nerve fibre length (CNFL), as a measure of corneal nerve health (Significant association; Pearson R=0.292, p=0.012). B. Correlation between tear NPY level and central corneal nerve fibre density (CNFD), as a measure of corneal nerve health (Significant association; Pearson R=0.294, p=0.012).

FIG. 4. Receiver operating characteristic ROC curves showing the diagnostic utility of tear NPY levels based on neuropathy criteria defined as (A) CNFL≤8.6 mm/mm² (to optimise specificity), (B) CNFL≤12.4 mm/mm² (to balance sensitivity and specificity), (C) CNFL≤15.3 mm/mm² (to optimise sensitivity), and (D) CNFD≤16.7 nerves/mm²

FIG. 5. Correlation between tear NPY level and sural sensory nerve amplitude in participants with type 1 diabetes (significant association, Pearson R=0.522, p=0.0005).

FIG. 6. Change in corneal sub-basal nerve density, measured using corneal nerve fibre length [CNFL] in the omega-3 and placebo groups over the study duration. Data are shown as A. central corneal nerve fibre length and B. change in central corneal nerve fibre length in the omega-3 supplementation (n=20) and placebo (n=20) groups over six months.

FIG. 7. Change in tear NPY levels in the omega-3 and placebo groups over the study duration. Data are shown as A. tear neuropeptide Y levels and B. change in tear neuropeptide Y levels in the omega-3 supplementation (n=20) and placebo (n=20) groups over six months.

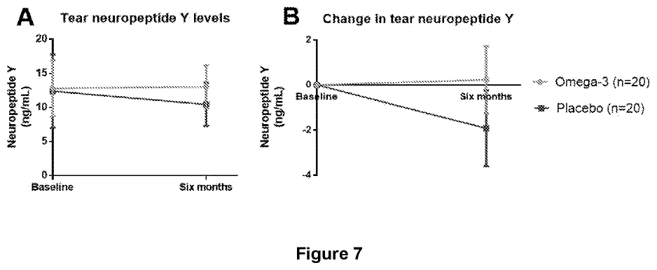

FIG. 8. Relationship between A. change in CNFL (mm/mm²) and B. change in CNFD (nerves/mm²), and baseline tear NPY levels (ng/mL) in individuals with type 1 diabetes treated with omega-3 fatty acids for six months.

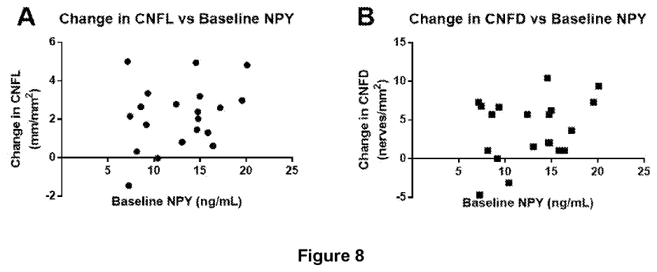

FIG. 9. Comparison of A. change in CNFL (mm/mm²) and B. change in CNFD (nerves/mm²) following 6-months' oral omega-3 fatty acid supplement intervention in individuals with type 1 diabetes with baseline tear NPY levels of ≤12 ng/mL and >12 ng/mL.

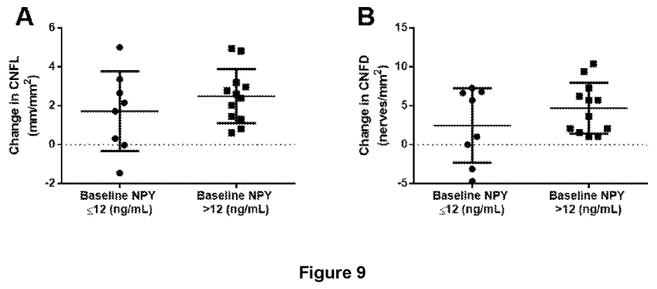

### DETAILED DESCRIPTION

Before describing the present invention in detail, it is to be understood that the terminology used herein is for the purpose of describing particular embodiments only and is not intended to be limiting.

As used in this specification and the appended claims, the singular forms “a”, “an” and “the” include plural referents unless the content clearly dictates otherwise.

The term “about”, as used herein, indicates the value of a given quantity can include quantities ranging within 10% of the stated value, or optionally within 5% of the value, or in some embodiments within 1% of the value.

Throughout the description and claims of the specification, the word “comprise” and variations of the word, such as “comprising” and “comprises”, is not intended to exclude other additives, components, integers or steps.

Unless specifically defined otherwise, all technical and scientific terms used herein shall be taken to have the same meaning as commonly understood by one of ordinary skill in the art.

All publications or patent applications or patents cited herein are entirely incorporated herein by reference.

A reference herein to a publication or patent application or patent or other matter which is given as prior art is not to be taken as admission that the document or matter (e.g., databases) was known or that the information it contains was part of the common general knowledge as at the priority date of any of the claims.

The present disclosure relates to methods of evaluation or diagnosis of the presence of or pre-disposition for a disease or condition, for example, corneal nerve damage or loss, corneal neuropathy, corneal neuropathic pain, corneal neuralgia, ocular disease, or peripheral neuropathy in a subject, as well as methods of prognosis of a subject having said disease or condition. The methods comprise determining the level of neuropeptide Y (NPY) or a fragment thereof in a tear fluid sample from the subject and comparing the level of the NPY to a reference level of expression of the NPY.

“Neuropeptide Y (NPY)” is a 36 amino acid peptide neurotransmitter and is a member of the pancreatic polypeptide class of neurotransmitters/neurohormones. NPY is derived from a larger, inactive precursor (SEQ ID NO:1). At least 6 NPY receptor subclasses have been identified and cloned to date. The NPY may have an amino acid sequence as shown in SEQ ID NO:2 or a fragment thereof.

As used herein in the context of proteins and peptides, the term “fragment” refers to smaller proteins or peptides derivable from larger proteins or peptides, which hence comprise a partial sequence of the larger protein or peptide. “Fragments” of the NPY preferably relate to fragments of at least 6 amino acids in length, most preferably at least 12 amino acid residues in length. Fragments of the NPY include, for example, NPY3-36, NPY13-26, NPY3-36, NPY1-30, NPY1-20, or NPY1-23 fragment. Such fragments are preferably detectable with assays as described herein. In some embodiments, fragments detectable with assays described herein include NPY3-36, NPY13-26, NPY3-36 and NPY1-30.

“Evaluation” in the context of the present disclosure relates to the assessment of the severity of the disease or condition in a subject.

“Diagnosis” in the context of the present disclosure relates to the recognition and (early) detection of a disease or condition in a subject and may also comprise differential diagnosis and pre-disposition of the subject to develop the disease or clinical condition. A diagnosis may include an assessment of the degree of disease/condition severity (e.g., “low” to “high”), current state of disease progression (e.g., “early”, “middle,” or “late” stages), or include a comparative assessment to an earlier diagnosis (e.g., “advancing”, “stable”, or in “remission”). For example, about <9.96 ng/ml NPY is indicative of more severe/late disease, about <12.35 ng/ml NPY is indicative of moderate disease, and about <15.23 ng/ml NPY is indicative of earlier/milder disease.

“Prognosis” denotes a prediction of how a subject's (e.g., a patient's) disease or condition will progress. This may include a prediction of the chance of recovery or the chance of an adverse outcome for said subject. Prognosis may also include a prediction of disease/condition response to treatment.

As used herein the term “subject” is intended to include all eukaryotic organisms shown to or expected to have NPY. In particular embodiments, the subject is a mammal, a human or nonhuman primate, a dog, a cat, a horse, a cow, other farm animals, or a rodent (e.g., mice, rats, guinea pig. etc.). The term “subject,” “patient,” and “individual” are used interchangeably herein.

The term “patient” as used herein includes a living human or non-human subject that is receiving medical care or that should receive medical care due to a disease or condition. This includes subjects with no defined illness or observable symptoms of a disease or condition who are being investigated for signs of pathology. Thus, the methods described herein are applicable to both, human and veterinary disease.

In some examples, the term “subject” refers to subjects at risk of (e.g., with systemic/ocular diseases that dispose to neuropathy) or with symptoms of (e.g., eye pain, etc.) corneal nerve damage or loss, corneal neuropathy, corneal neuropathic pain, corneal neuralgia, ocular disease, or peripheral neuropathy.

In some examples, the subject is identified as being at risk of exposure to corneal nerve damage or loss that could result from disease, trauma, or a medical procedure. In some examples, the subject is identified as being at risk of exposure to corneal nerve damage or loss that could result from diabetes mellitus, neurotrophic keratitis, neurotrophic keratopathy, herpes simplex keratitis, herpes zoster keratitis, trigeminal nerve damage, ocular or head surgery, ocular or head trauma, aneurysm, intracranial neurologic disease, keratorefractive procedures, photorefractive keratectomy (PRK), laser in situ keratomileusis (LASIK), ocular surface disease, a non-ophthalmic disorder (such as COVID-19 infection or auto-immune disease), a non-ophthalmic procedure, peripheral neuropathy, or diabetic neuropathy. In one example of the disclosure, the corneal nerve damage or loss results from peripheral neuropathy or diabetic neuropathy or related condition.

In some examples, the methods include the step of identifying a subject with a sign or symptom of corneal nerve damage or loss. For example, a sign of corneal nerve damage or loss is a decrease of corneal innervation or sensation, a reduction in the number of nerve fibres or bundles innervating the cornea, death of neurons innervating the cornea, a decrease or loss of neurotransmitter release, a decrease or loss of nerve growth factor release, abnormal tearing reflexes, abnormal blink reflexes, abnormal nerve morphology, appearance of abnormal nerve sprouts, corneal microneuromas or neuromas, abnormal nerve tortuosity, increased bead-like nerve formations, thinning of nerve fibre bundles, or thickening of nerve fibre bundles. For example, a symptom of corneal nerve damage or loss is abnormal tear production or eye discomfort, abnormal blinking, and difficulty or loss of ability to focus, decreased or lost visual acuity, or decreased or lost corneal sensitivity.

In some examples, the methods of the disclosure further comprise administering to a subject identified as having corneal nerve damage or loss of nerves or a predisposition thereto, or suffering from or is at risk of corneal neuropathy, corneal neuropathic pain, or corneal neuralgia, for example, a therapeutically or prophylactically effective amount of a treatment for preventing, inhibiting, or reducing nerve damage or loss, or for ameliorating, preventing, inhibiting, or reducing corneal neuropathy, corneal neuropathic pain, or corneal neuralgia in the subject. Such a treatment may be selected from, for example, one or more anti-inflammatory agents, neuroprotective agents, neuroactive agents (e.g., lacosamide), neuroregenerative agents (e.g., nerve growth factor), neuromodulation, immunomodulatory agents, biologics (including, blood-derived topical preparations, and devices such as PROKERA), vitamins and/or nutritional supplements (e.g., omega-3 fatty acids), anti-glycaemic agents, neurostimulation, ocular protection (e.g., contact lenses), environmental modification (e.g., use of a humidifier), pain therapies (including drugs, devices, etc.), dietary modification, or combinations thereof.

In other or further examples, the methods of the disclosure further comprise administering to a subject identified as suffering from or being at risk of developing peripheral neuropathy (for example, corneal neuropathy, an ocular manifestation of peripheral neuropathy in, for example, individuals with diabetes (e.g., diabetes mellitus) an effective amount, for example, a therapeutically or prophylactically effective amount of a treatment for ameliorating, preventing, inhibiting, or reducing diabetic peripheral neuropathy in the subject. Such a treatment may be selected from, for example, one or more anti-glycaemic agents, aldose-reductase inhibitors, anti-oxidant therapies (e.g., alpha-lipoic acid), metabolic agents (e.g., actovegin), vaso-active compounds (e.g., ACE inhibitors, C-peptide), lipid-active agents (e.g., fenofibrate), neuroregenerative agents (e.g., nerve growth factor), neurostimulation, vitamins and/or nutritional supplements (e.g., omega-3 fatty acids, benfotiamine), pain management approaches (e.g., serotonin-norepinephrine reuptake inhibitors, tricyclic antidepressants, anti-epileptics), topical agents (e.g., topical capsaicin, anaesthetics), alternative therapies (e.g., acupuncture), or combinations thereof. Alternatively, or in addition, lifestyle changes (e.g., maintaining blood glucose in target ranges, dietary changes, exercise and weight maintenance) may be useful for reducing neuropathy progression (once identified in a subject).

As used herein, the terms “treating”, “treat” or “treatment” refers to reducing or eliminating at least one symptom of the disease or condition or to inhibit or slow progression of the disease or condition.

As used herein, the terms “preventing”, “prevent” or “prevention” includes providing prophylaxis with respect to occurrence or recurrence of the disease or condition. An individual may be predisposed to or at risk of developing the disease or disease relapse but has not yet been diagnosed with the disease or the relapse.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment of the disease or condition. The effective amount may vary according to the disease or condition to be treated or factor to be altered and also according to the weight, age, racial background, sex, health and/or physical condition and other factors relevant to the subject being treated. Typically, the effective amount will fall within a relatively broad range (e.g., a “dosage” range) that can be determined through routine trial and experimentation by a medical practitioner. Accordingly, this term is not to be construed to limit the disclosure to a specific quantity. The effective amount can be administered in a single dose or in a dose repeated once or several times over a treatment period.

A “therapeutically effective amount” is at least the minimum concentration required to effect a measurable improvement of a particular disease or condition or to inhibit or delay the onset of one or more detectable symptoms of a disease or condition or a complication thereof. A therapeutically effective amount herein may vary according to factors such as the disease state, age, sex, and weight of the patient. A therapeutically effective amount is also one in which any toxic or detrimental effects of the treatment are outweighed by the therapeutically beneficial effects.

As used herein, “corneal neuropathy” is broadly defined by a reduction in corneal nerves and/or a loss of typical corneal sensation. Typical signs of corneal neuropathy, which indicates corneal nerve damage or loss, are a reduction in corneal nerve fibre length (CNFL) and/or corneal nerve fibre density (CNFD), measured using a technique such as in vivo confocal microscopy. These are quantitative parameters of the corneal nerve anatomical architecture. CNFL is generally considered the more robust measurement, and preferred as an outcome in clinical trials, although both parameters are typically correlated (e.g., using the image analysis software ACCMetrics). In one example of the disclosure, the corneal neuropathy results from diabetes mellitus. In other examples of the disclosure, the corneal neuropathy results from small fibre neuropathy (non-specific), post-refractive surgery (e.g., post-LASIK, post-PRK), COVID-19 induced corneal nerve damage (including long-COVID), autoimmune nerve damage (e.g., Sjogren's syndrome), infection (e.g., acanthamoeba with nerve damage related to keratoneuritis), post-viral infection, toxicity-induced (e.g., post-chemotherapy), post-corneal surgery leading to corneal nerve damage (such as corneal collagen cross-linking), post-trauma, recurrent corneal erosions, corneal ectasias, corneal dystrophies, neurotrophic keratopathy, central nervous system neurodegenerative disorders (e.g., Alzheimer's disease), contact lens-induced, trigeminal neuralgia, etc. “Corneal neuropathy” can be used interchangeably with corneal “neuropathic pain” and “corneal neuralgia”.

As used herein, “peripheral neuropathy” can be, for example, a result of diseases of the nerves (primary neuropathy) and neuropathy that is caused by systemic disease (secondary neuropathy), such as but not limited to diabetic neuropathy, Herpes Zoster (shingles)-related neuropathy, uraemia-associated neuropathy, amyloidosis neuropathy, HIV sensory neuropathies, hereditary motor and sensory neuropathies (HMSN), hereditary sensory neuropathies (HSNs), hereditary sensory and autonomic neuropathies, hereditary neuropathies with ulcero-mutilation, nitrofurantoin neuropathy, tumaculous neuropathy, neuropathy caused by nutritional deficiency and neuropathy caused by kidney failure. Other causes include repetitive activities such as typing or working on an assembly line, medications known to cause peripheral neuropathy, such as several AIDS drugs (DDC and DDI), antibiotics (metronidazole, an antibiotic used for Crohn's disease, isoniazid used for tuberculosis), gold compounds (used for rheumatoid arthritis), some chemotherapy drugs (such as vincristine and others) and many others. Chemical compounds are also known to cause peripheral neuropathy including alcohol, lead, arsenic, mercury and organophosphate pesticides. Some peripheral neuropathies are associated with infectious processes (such as Guillian-Barre syndrome). In one example of the disclosure, the peripheral neuropathy is an abnormal pain perception diabetic neuropathy or related condition.

The term “sample” as used herein refers to a tear fluid sample obtained for the purpose of diagnosis, prognosis, or evaluation of a subject. The sample may be treated, for example, diluted (e.g., up to fold of 40×), concentrated, fractionated, or purified (e.g., partially purified) prior to analysis. The undiluted sample may be between about 0.2 μL and about 5 μL, for example, about 1.25 μL. The skilled person will appreciate that the sample volume needed will depend on the detection assay used (e.g., based on its sensitivity).

The term “level” as used herein relates to the concentration (preferably expressed as weight/volume; w/v) of NPY in the sample.

Any suitable “assay” or “diagnostic assay” can used to determine the level of NPY in the sample. Such an assay may be based on the binding of an analyte (i.e., NPY) to be detected to one or more capture probes with a certain affinity. Concerning the interaction between capture molecules and target molecules or molecules of interest, the affinity constant is preferably greater than 10⁻⁸ M.

In the context of the present disclosure, “capture molecules” are molecules which may be used to bind target molecules or molecules of interest, i.e., analytes (i.e., in the context of the present disclosure, NPY), from a sample. Capture molecules must thus be shaped adequately, both spatially and in terms of surface features, such as surface charge, hydrophobicity, hydrophilicity, presence or absence of Lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present invention, capture molecules may for instance be selected from the group comprising a nucleic acid molecule, a carbohydrate molecule, a PNA molecule, a protein, an antibody, a peptide or a glycoprotein. Preferably, the capture molecules are antibodies, including fragments thereof with sufficient affinity to a target or molecule of interest, and including recombinant antibodies or recombinant antibody fragments, as well as chemically and/or biochemically modified derivatives of said antibodies or fragments.

The preferred detection methods comprise immunoassays in various formats such as for instance radioimmunoassay (RIA), chemiluminescence- and fluorescence-immunoassays, Enzyme-linked immunoassays (ELISA), Luminex-based bead arrays, protein microarray assays, and rapid test formats such as for instance immunochromatographic strip tests. Other detection methods include proteomic/mass-spectrometry techniques known to those skilled in the art and may be used to quantify levels of tear NPY.

The assays can be homogenous or heterogeneous assays, competitive and non-competitive assays. In a particularly preferred embodiment, the assay is in the form of a sandwich assay, which is a non-competitive immunoassay, wherein the molecule to be detected and/or quantified is bound to a first antibody and to a second antibody. The first antibody may be bound to a solid phase, e.g., a bead, a surface of a well or other container, a chip or a strip, and the second antibody is an antibody which is labeled, e.g., with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005); Hultschis et al., Curr, Opin, Chem, Biol. (2006) 10(1):4-10).

In one example, the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

The labelling system may comprise rare earth cryptates or rare earth chelates in combination with fluorescence dye or chemiluminescence dye, in particular a dye of the cyanine type.

In the context of the present disclosure, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, CY7, Xanthen, 6-Carboxy-2′,4′,74,7-hexachlorofluorescein (HEX), TET, 6-Carboxy-4′,5′-dichloro-2′,7′-dimethoxyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-Carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present disclosure, chemiluminescence based assays comprise the use of dyes, based on the physical principles described for chemiluminescent materials in for example, Kirk-Othmer, Encyclopedia of chemical technologv, 4th ed., executive editor, J. I. Kroschwitz; editor, M. Howe-Grant, John Wiley & Sons, 1993, vol. 15, p. 518-562 including citations on pages 551-562. Preferred chemiluminescent dyes are acridiniumesters.

The term “correlating” or “correlated” as used herein refers to comparing the level of the NPY in the sample to its amount in subjects known to have, or known to be at risk of, a given disease or condition, or subjects known to respond or not respond to a given treatment.

The level of NPY in a sample can be compared to a level known to be indicative of an abnormal state. In some embodiments, the level has been correlated with a diagnosis; that is, the skilled person can use the level to determine whether the subject has a disease or condition or a pre-disposition thereto and respond accordingly. Alternatively, the level can be compared to a level indicative of a normal state. In some embodiments, the level has been correlated with the absence of disease or a condition. In other embodiments, the level can be compared to a level of NPY in a sample taken from the subject at an earlier timepoint, to, for example, monitor disease progression or treatment efficacy. In some embodiments, the level can be compared to a baseline level of NPY in a sample taken before initiation of treatment or following a round of treatment. In other embodiments, the level has been correlated with a response or non-response to a given treatment.

The sensitivity and specificity of a diagnostic and/or prognostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e., apparently healthy) and “disease” populations. For any particular marker, a distribution of NPY levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results do not necessarily give an accurate number. As long as one can rank results, one can create a ROC curve. For example, results of a test on “disease” samples might be ranked according to degree (e.g., 1=low, 2=normal, and 3=high). This ranking can be correlated to results in the “normal” population, and a ROC curve created. These methods are well known in the art. Preferably, ROC curves result in an AUC of greater than about 0.5, more preferably greater than about 0.7, still more preferably greater than about 0.8, even more preferably greater than about 0.85, and most preferably greater than about 0.9. The term “about” in this context refers to +/−5% of a given measurement.

The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the discriminative capacity of the test.

In certain embodiments, NPY as a marker exhibits at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

Preferred cut-off values are for instance the 90th, 95th or 99th percentile of a normal population. By using a higher percentile than the 75th percentile, one reduces the number of false positive subjects identified, but one might miss to identify subjects, who are at moderate, albeit still increased risk. Thus, one might adopt the cut-off value depending on whether it is considered more appropriate to identify most of the subjects at risk at the expense of also identifying “false positives”, or whether it is considered more appropriate to identify mainly the subjects at high risk at the expense of missing several subjects at moderate risk.

The present disclosure finds a significant positive correlation between the level of NPY and each of CNFL and CNFD.

In a preferred embodiment of the disclosure, the subject is diagnosed with having corneal nerve damage or loss (or corneal neuropathy, corneal neuropathic pain, or corneal neuralgia), when the determined NPY level is correlated with a diagnostic threshold level of corneal nerve fibre length (CNFL) and/or corneal nerve fibre density (CNFD) in subjects who are susceptible to, or have peripheral nerve damage (e.g., diabetic subjects).

In some examples, the diagnostic threshold level of CNFL is between about 8 and about 16 mm/mm², between about 8.5 and about 15.5 mm/mm² between about 8.6 and about 15.3 mm/mm², between about 10 and about 14 mm/mm², between about 10.5 and about 13.5 mm/mm², between about 11 and about 13 mm/mm², between about 11.5 and about 12.5 mm/mm², about 12.5, about 12.4, or about 12.3 mm/mm² as determined using the image analysis software ACCMetrics (developed by researchers at the University of Manchester and Weill-Cornell University Qatar) for quantifying corneal in vivo confocal microscopy images obtained using the Heidelberg HRT III corneal confocal microscope (Dabbah et al., Automatic Analysis of Diabetic Peripheral Neuropathy using Multi-scale Quantitative Morphology of Nerve Fibres in Corneal Confocal Microscopy Imaging, Journal of Medical Image Analysis (2011) 15(5):738-747; Dabbah et al., Dual-model automatic detection of nerve-fibres in corneal confocal microscopy images, Med. Image Comput. Assist. Interv. (2010) 13:300-307; Chen et al., An Automatic Tool for Quantification of Nerve Fibres in Corneal Confocal Microscopy Images, IEEE Transactions on Biomedical Engineering (2016) 786-794;
https://weillcornell.azl.qualtrics.com/jfe/form/SV_6o2ji0suM4jQinb_).

In some or further examples, the diagnostic threshold level of CNFD is between about 5.5 and about 18 mm/mm², between about 6 and about 17 mm/mm² between about 6.24 and about 16.7 mm/mm², between about 15 and about 18.5 mm/mm², between about 15.5 and about 18 mm/mm², between about 16 and about 17.5 mm/mm², between about 16 and about 17 mm/mm², between about 16.5 and about 17 mm/mm², about 16.5, about 16.6, or about 16.7 mm/mm² as determined using the image analysis software ACCMetrics.

In one example, the diagnostic threshold level of CNFL and CNFD is between about 8 and 18 mm/mm², between about 8.5 and about 17 mm/mm² between and about 8.6 and 16.7 mm/mm², between about 10 and 18 mm/mm², between and 10.5 and 18 mm/mm², between about 11 and about 18 mm/mm² between about 11.5 and about 17.5 mm/mm², between about 12 and about 17 mm/mm², or between about 12.3 and about 16.7 mm/mm² as determined using the image analysis software ACCMetrics.

In some examples, the predetermined threshold level of NPY that correlates with a diagnostic threshold level of CNFL is between about 9 and about 18 ng/mL, between about 8.5 and about 17.8 ng/mL, between about 8.6 and about 17.7 ng/mL, between about 14 and about 17 ng/mL, between about 14.5 and about 16.5 ng/mL, between about 15 and about 16 ng/mL, between about 15 and about 15.5 ng/mL, about 15.1, about 15.2, or about 15.3 ng/mL.

In some examples, the predetermined threshold level of NPY that correlates with a diagnostic threshold level of CNFD is between about 6 and 12.5 ng/mL, between 6.1 and 12.4 ng/mL, between about 6.24 and about 12.35 ng/mL, between about 11 and about 14 ng/mL, between about 11.5 and about 13.5 ng/mL, between about 12 and about 13 ng/mL, between about 12 and about 12.5 ng/mL, about 12.1, about 12.2, about 12.3, or about 12.4 ng/mL.

In some embodiments of the disclosure, the subject is diagnosed with having diabetic peripheral neuropathy when the determined NPY level is correlated with a predetermined threshold level that is diagnostic for diabetic sensorimotor polyneuropathy. In some examples, a sural amplitude measure of ≤6 uV is used as the diagnostic threshold for diabetic sensorimotor polyneuropathy and this correlates with between about 8 to about 14 ng/mL NPY in diabetic subjects. For example, between about 8.5 and about 13.9 ng/mL, about 8.6 and about 13.8 ng/mL, about 8.7 and about 13.6 ng/mL NPY. In some examples, the predetermined threshold level of NPY that correlates with a diagnostic threshold level of diabetic sensorimotor polyneuropathy is between about 8.5 and about 12.5 ng/mL, between about 9 and about 12.5 ng/mL, between about 9.1 and about 12.2 ng/mL, about 9.1 ng/mL, or about 12.2 ng/mL (for increased specificity).

In some examples, the subject is diagnosed with corneal nerve damage or loss, corneal neuropathy, corneal neuropathic pain, corneal neuralgia, ocular disease, or peripheral neuropathy when said determined NPY level is lower than a predetermined threshold level. Preferably, the predetermined threshold level is between about 9 and about 15 ng/mL, for example, about 9.5 to about 14.8 ng/mL. In a preferred embodiment the subject is diagnosed with having corneal nerve damage or loss, corneal neuropathy, corneal neuropathic pain, corneal neuralgia, ocular disease, or peripheral neuropathy when said determined NPY level is lower than about 12.5 ng/mL.

In some embodiments, the level of NPY in the sample is combined with one or more clinical parameters selected from the group consisting of age, gender, duration of diabetes, HbA1c, fasting C-peptide, blood urea nitrogen, 24-h urinary protein, 24-h urinary protein excretion rate, vibration perception threshold and retinal vessel geometric parameters, including CRAE, CRVE, DFa, DFv, BCa, BCV, BAa, BAv.

The term “combined” or variations such as “combination” or “combining” is defined as a possible selection of a certain number of parameters and the arrangement of these parameters into specified groups using a mathematical algorithm (e.g. deviation or ratio). For example, a ratio can be calculated between the level of a biomarker (e.g., NPY) in a sample taken from a subject and the level of the same biomarker in a sample taken from the subject at an earlier time point to provide a baseline level (e.g., before the start of treatment). Moreover, a deviation can be calculated between the level of a biomarker in a sample taken from a subject and the level of the same biomarker in a sample taken from the subject at an earlier time point. It is also encompassed herein, that a ratio between different biomarkers can be calculated. For example, the ratio can be calculated between biomarker levels measured in samples taken from the subject at the same time point or at different time points.

In some embodiments, the level of the at least one biomarker can be combined as continuous or categorical variable.

The term “score” in the context of the present disclosure refers to a rating, expressed numerically, based on the specific achievement or the degree to which certain qualities or conditions (e.g. the level of NPY) are present in the sample.

In some embodiments of the disclosure, the level of NPY in the sample can be used to evaluate the severity of the disease or condition. For example, the level of NPY in the sample can be used to evaluate the severity of corneal neuropathy, corneal neuropathic pain, or corneal neuralgia.

In some embodiments of the disclosure, the subject is stratified as having early, moderate or severe corneal nerve damage or loss when the determined NPY level is correlated with a predetermined threshold level of CNFL and/or CNFD that is diagnostic for differing degrees of corneal nerve damage or loss. In some examples, a CNFL of about 4 to about 8.6 mm/mm², is used as the diagnostic threshold for severe corneal nerve loss or damage and this correlates with between about 5 to about 11 ng/mL NPY. In other examples, a CNFL of about 4 to about 8.6 mm/mm², is used as the diagnostic threshold for moderate corneal nerve loss or damage and this correlates with between about 8 to about 15 ng/mL NPY. In other examples, a CNFL of about 12.5 to about 15.3 mm/mm², is used as the diagnostic threshold for early corneal nerve loss or damage and this correlates with between about 11 to about 20 ng/mL NPY. In some embodiments, the CNFL and/or CNFD value is used as a surrogate marker of peripheral neuropathy more generally.

The term “treatment stratification” in the context of the present disclosure refers to the choice and/or adjustment of a therapeutic treatment of the subject. For example, subjects having a NPY of greater than about 12 ng/ml may be considered responsive to treatment, for example, to an anti-inflammatory and/or neuroprotective intervention.

The term “risk stratification” in the context of the present disclosure refers to a probability of specified outcomes for a subject. For example, subjects having a NPY of less than about 12.5 ng/ml may be at risk for corneal nerve damage or loss, corneal neuropathy, corneal neuropathic pain, corneal neuralgia, ocular disease, or peripheral neuropathy.

The skilled person will appreciate that the diagnostic levels given herein have been calculated based on a specific assay. And that such absolute levels may change depending on how NPY levels are detected in the tear fluid sample. It would be routine in the art for the skilled person to calculate new diagnostic levels of tear NPY detected using a different assay, now that NPY has been shown to be a diagnostic biomarker of corneal nerve damage or loss, corneal neuropathy, corneal neuropathic pain, corneal neuralgia, ocular disease and peripheral neuropathy.

### EXAMPLES

### Methods and Materials

### Participants:

Participants were examined at the Department of Optometry and Vision Sciences, University of Melbourne between March 2018 and March 2020.

Participants included 51 adults aged 18 years or over with either type 1 (n=44) or type 2 (n=7) diabetes, and 22 healthy controls with no uncontrolled systemic or ocular conditions. Participant exclusion criteria included i) a known bleeding disorder; ii) a history of rigid contact lens wear; iii) a known allergy or previous hypersensitivity to any ocular agents required for the study (i.e., topical ocular anaesthetic, sodium fluorescein dye, lissamine green dye); iv) any of: active ocular inflammation or infection, history of recurrent herpetic keratitis or active disease within six months of baseline, corneal disorders or abnormalities that may affect the integrity of the corneal nerves; v) any of: active ocular infection or active ocular surface inflammation except dry eye; vi) a diagnosis of neuropathy from any cause other than diabetes; vii) best spectacle corrected visual acuity of worse than 6/12 in either eye; viii) women who were pregnant or breastfeeding. Participants who wore soft contact lenses were asked to refrain from lens wear for at least 24 hours before examinations. Participants using lubricating eye drops were asked to not instill these for at least two hours before examination procedures.

### Study Subpopulation (Therapeutic Assessment)

From the study cohort, 40 participants with type 1 diabetes were enrolled in the randomised controlled trial (RCT) “Investigating the Neuroprotective Effect of Oral Omega-3 Fatty Acid Supplementation in Type 1 Diabetes (nPROOFS1)” (registry number ACTRN12618000705280) and completed said trial (Britten-Jones et al., Investigating the neuroprotective effect of Oral Omega-3 Fatty acid Supplementation in type 1 diabetes (nPROOFS1): a randomised, placebo-controlled trial, Diabetes (2021) 70:1794-1806).

The 40 individuals were randomly assigned (1:1) to oral long-chain omega-3 fatty acid supplements (1800 mg/day; n=20) or placebo (olive oil) supplements (600 mg/day; n=20) for 180 days.

Study assessments included that of described below, performed at the University of Melbourne at baseline, at days 30±14, 90±14, and 180±24. Non-stimulated (basal) tear samples were collected at baseline (day 0) and endpoint (day 180±24) and analysed using the ELISA methods described below.

At baseline (day 0) and endpoint (day 180±24), participants additionally undertook study visits to evaluate peripheral small and large nerve fibre function at St Vincent's Hospital, Melbourne. Study efficacy outcomes included change in peripheral small nerve fibre function, measured using cutaneous silent periods and quantitative sudomotor axonal reflex test, and large nerve fibre function, measured using nerve conduction studies.

### Ocular Assessments

Assessments of ocular surface health included dry eye symptoms, tear osmolarity, slit lamp examination and grading, tear break-up time, and ocular surface staining. Corneal nerve function was assessed using non-contact corneal aesthesiometry, with both room temperature stimuli (23 to 24° C.) and cooled stimuli (18 to 19° C.) (Britten-Jones et al., Investigating the neuroprotective effect of Oral Omega-3 Fatty acid Supplementation in type 1 diabetes (nPROOFS1): a randomised, placebo-controlled trial, Diabetes (2021) 70:1794-1806).

Corneal nerve structure was evaluated in the right eye of participants using in vivo confocal microscopy (IVCM), which is a validated surrogate marker of peripheral nerve integrity in other regions of the body. Corneal sub-basal nerve plexus parameters were quantified using gold-standard, automated software (ACCMetrics; Dabbah et al., Automatic analysis of diabetic peripheral neuropathy using multi-scale quantitative morphology of nerve fibres in corneal confocal microscopy imaging. Med Image Anal. (2011) 15:738-747). The corneal sub-basal nerve plexus parameters that were quantified are described in Table 1.

TABLE 1
Main corneal sub-basal nerve plexus parameters quantified using ACCMetrics

| Parameter | Unit | Definition |
|---|---|---|
| Corneal nerve fibre | mm/mm² | The total length of all nerve fibres in the |
| length (CNFL) | | image capture frame |
| Corneal nerve fibre | fibres/mm² | The total number of main fibres divided by |
| density (CNFD) | | the area of the image frame |
| Corneal nerve | branches on main | The total number of main nerve branches |
| branch density | fibre/mm | that stem directly from a nerve fibre, |
| (CNBD) | | divided by the area of the image frame |
| Corneal nerve total | total branches/mm | The total number of branches within the |
| branch density | | area of the image frame |
| (CTBD) | | |

### Tear Neuropeptide Y Analysis

Non-stimulated (basal) tear samples were collected using microcapillary tubes by capillary flow from the inferior lateral meniscus of the eye. About 5 μL were collected from each eye; tears were stored at −80° C. until analysis. Neuropeptide analysis was performed using tears collected from the right eye. A minimum of 1.25 μL of tear volume per analyte (to obtain a maximum dilution fold of 33%) was used for the analyses.

Enzyme-linked immunosorbent assay (ELISA) was performed to measure the NPY concentrations in the tear samples according to the manufacturer's instructions for the commercial NPY ELISA kit (Cat. EK-049-03CE, Phoenix Pharmaceuticals, USA). Standard NPY dilutions with a concentration of 0.01, 0.1, 1, 10, 100, 1000 ng/ml were prepared for standard curve plot. Up to 5 μL of tear samples were diluted into 100 μL sample dilutions using assay buffer. 50 μL of standard dilution, sample dilution or positive control was added into each well in duplicate, followed by 25 μL of primary antibody and 25 μL of biotinylated peptide into each well. The immunoplate was sealed and incubated at room temperature for two hours. After four rounds of washing using assay buffer, 100 μL of streptavidin horseradish peroxidase (to catalyse the substrate solution) was added into each well, followed by a 1-hour incubation at room temperature. Then the immunoplate was washed 4 times and 100 μL of substrate solution was added into each well. After incubating for another 1 hour at room temperature, 100 μL of 2M HCl was added into each well to stop the reaction. The optical density absorbance reading was measured at 450 nm using Bio Tek Epoch2 Microplate Spectrophotometer (BioTek, USA). The standard curve fitting was performed using 4 parameter logistics in Prism software (GraphPad, USA), and the NPY concentrations in the tear samples were determined after multiplying the dilution factors.

### Statistical Analysis

Data normality was assessed using the Shapiro-Wilk test. Descriptive statistics are summarised as mean±standard deviation (SD) for normally distributed data, or median (inter-quartile range, IQR) for non-normally distributed data. Inter-group comparisons were analysed using either the Welch t-test for normally distributed variables or the Mann-Whitney-U test for non-normally distributed variables. Fisher's exact test (two-tailed) was used to compare frequencies of categorical variables between groups. The association between corneal structural parameters and tear NPY levels was evaluated using Pearson's correlation coefficient (R) for normally distributed data and Spearman's correlation coefficient (p) for non-normally distributed data.

### Results

### Participant Characteristics

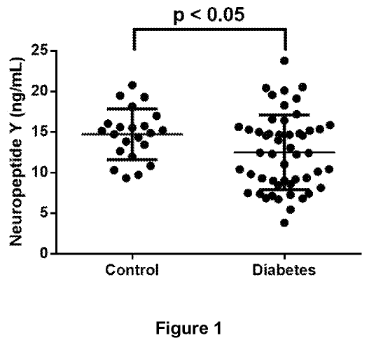

Participants with and without diabetes were similar in age, and both groups had similar gender distributions. Consistent with the presence of early-stage corneal neuropathy, central corneal nerve structural parameters (e.g., corneal nerve fibre length [CNFL] and corneal nerve fibre density [CNFD]) were lower in participants with diabetes than those without (FIG. 1). Central corneal sensitivity was also significantly lower (i.e., thresholds were higher) in the diabetes subjects.

TABLE 2
Participant demographics

| | | | |
|---|---|---|---|
| Controls with no | | | |
| peripheral | Diabetes | | |
| neuropathy | participants | Overall | p- |
| (n = 22) | (n = 51) | (n = 73) | value* |

Demographics

| | | | | |
|---|---|---|---|---|
| Age, years | 51 (28-64) | 44 (28-65) | 45 (27-64) | 0.75 |
| Sex, number of females, n (%) | 15 (68%) | 25 (49%) | 40 (55%) | 0.20 |
| Type-1 diabetes, n (%) | — | 44 | — | — |
| Diabetes duration, years | — | 16 (6.50-26) | — | — |

Ocular surface parameters

| | | | | |
|---|---|---|---|---|
| OSDI score, /100 | 5.4 (0-12.0) | 6.3 (2.1-10.0) | 6.3 (2.1-10.4) | 0.95 |
| Tear osmolarity, mOsmol/L | 301 (294-304) | 3.2 (295-311) | 302 (295-309) | 0.34 |
| Conjunctival redness, /4.0 | 0.82 (0.70-1.01) | 0.80 (0.71-1.06) | 0.80 (0.62-1.05) | 1.0 |
| Limbal redness, /4.0 | 0.32 (0.21-0.62) | 0.45 (0.28-0.88) | 0.40 (0.25-0.80) | 0.14 |
| TBUT, seconds | 10.24 (8.78-11.63) | 7.59 (5.82-8,91) | 8.81 (6.97-10.96) | 0.003 |
| Corneal staining, /4.0 | 0.17 (0.10-0.40) | 0.15 (0.0-0.55) | 0.15 (0.0-0.55) | 0.63 |
| Conjunctival staining, /4.0 | 0.16 (0.03-0.34) | 0.05 (0.0-0.28) | 0.12 (0.0-0.30) | 0.37 |

Central corneal sensitivity

| | | | | |
|---|---|---|---|---|
| Room-temperature stimuli | 0.28 (0.18-0.37) | 0.45 (0.30-0.74) | 0.38 (0.28-0.55) | <0.001 |
| (mbar) | | | | |
| Cooled stimuli (mbar) | 0.15 (0.05-0.26) | 0.42 (0.25-0.60) | 0.32 (0.15-0.55) | <0.001 |

Central corneal nerve structure

| | | | | |
|---|---|---|---|---|
| Central CNFL (mm/mm²) | 14.91 ± 3.06 | 12.21 ± 3.18 | 13.01 ± 3.36 | 0.001 |
| Central CNFD (nerves/mm²) | 25.01 ± 6.13 | 20.56 ± 7.01 | 21.90 ± 7.02 | 0.009 |
| Central CNBD (branches/mm²) | 36.39 ± 13.90 | 22.48 ± 10.41 | 26.67 ± 13.15 | <0.001 |
| Central CTBD (total | 52.87 ± 20.53 | 33.13 ± 14.09 | 39.08 ± 18.54 | <0.001 |
| branches/mm²) | | | | |

*p-value calculated using Fisher exact test (two tailed) for sex, Welch t-test for central corneal nerve structure, and Mann-Whitney U-test for age and central corneal sensitivity.

Tear NPY Levels in Individuals with, or are at Risk of, Neuropathy:

The tear NPY concentrations (levels) were significantly lower in participants with diabetes compared with those without (FIG. 1; mean±SD: diabetes: 12.50±0.64 vs control: 14.72±0.67 ng/mL; p=0.02).

The NPY concentrations in tears were significantly lower in those with high likelihood of neuropathy (CNFD≤16.7 mm/mm; mean±SD: 10.78±0.81 ng/mL. CNFL diagnostic criterion derived from Perkins et al., Corneal confocal microscopy for identification of diabetic sensorimotor polyneuropathy: a pooled multinational consortium study. Diabetologia (2018) 61:1856-1861) compared to those without (CNFD>16.7 mm/mm; mean±SD: 14.07±0.58 ng/mL; p=0.0021).

### Relationship Between Tear NPY Level and Corneal Nerve Structural Parameters

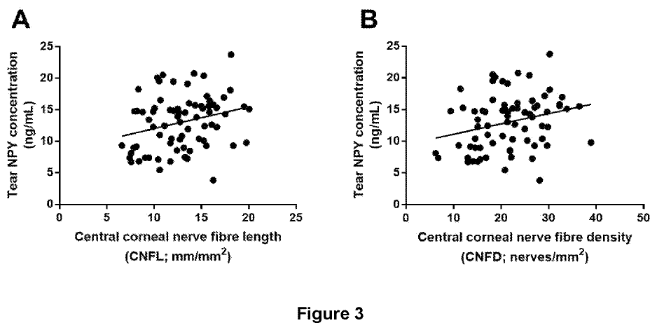

There was a significant positive correlation between tear NPY concentration (level) and two key central corneal nerve anatomical parameters (corneal nerve fibre length (CNFL), FIG. 3A and corneal nerve fibre density (CNFD), FIG. 3B). These data indicate a relationship between the severity of corneal nerve damage or loss and tear film levels of NPY (i.e., individuals with lower tear NPY levels generally have fewer corneal nerves).

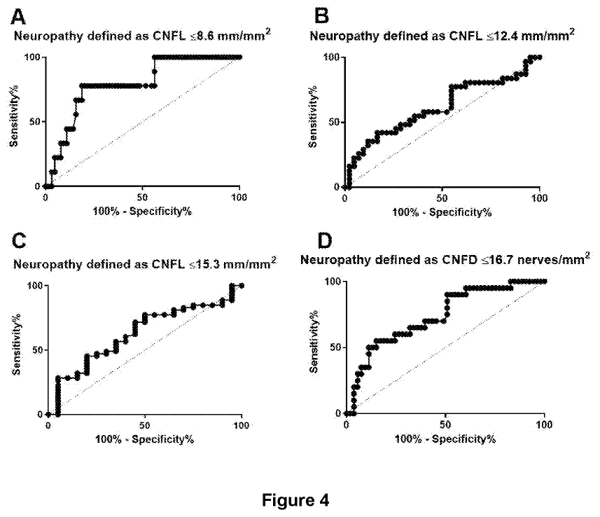

Based on the work of Perkins et al. (Corneal confocal microscopy for identification of diabetic sensorimotor polyneuropathy: a pooled multinational consortium study. Diabetologia (2018) 61:1856-1861) to define different CNFL and CNFD cut-offs to identify the presence of peripheral neuropathy (which includes corneal neuropathy), Receiver Operator Characteristics (ROC) curves were plotted (FIG. 4) using the following CNFL thresholds: ≤8.6 mm/mm² (to optimise specificity), ≤12.4 mm/mm² (to balance sensitivity/specificity), ≤15.3 mm/mm² (to optimise sensitivity); and CNFD≤16.7 nerves/mm² (to balance sensitivity/specificity).

If peripheral neuropathy (including corneal neuropathy) is defined on the basis of:
a CNFL of ≤8.6 mm/mm², using a criterion where tear NPY is <9.5 ng/ml gives 78% sensitivity (95% CI 40% to 97%) and 82% specificity (95% CI 70% to 90%) to identify this state.
a CNFL of ≤12.4 mm/mm², using a criterion where tear NPY is <12.4 ng/ml gives 51% sensitivity (95% CI 33% to 70%) and 67% specificity (95% CI 50% to 80%) to identify this state.
a CNFL of ≤15.3 mm/mm², using a criterion where tear NPY is <15.3 ng/ml gives 77% sensitivity (95% CI 64% to 88%) and 50% specificity (95% CI 27% to 73%) to identify this state.
a CNFD of ≤16.7 nerves/mm², using a criterion where tear NPY is <12.35 ng/ml gives 65% sensitivity (95% CI 41% to 85%) and 68% specificity (95% CI 54% to 80%) to identify this state.

### Relationship Between Tear NPY and Peripheral Nerve Sensory Function

In a clinical population with type 1 diabetes (n=20; derived from the baseline data from the RCT), there was a significant positive relationship between tear NPY levels and sural sensory nerve amplitude (FIG. 4, p=0.0005), which is a standard measures of peripheral nerve function (Malik et al., Sural nerve pathology in diabetic patients with minimal but progressive neuropathy. Diabetologia (2005) 48:578-585). As such, people with low levels of tear NPY had poorer peripheral sensory nerve function, as measured using nerve conduction tests.

### Tear NPY Levels as a Marker for Assessing Treatment Response

Of the 40 individuals with type 1 diabetes who were enrolled into a randomised controlled trial, 20 were randomly assigned (1:1) to oral long-chain omega-3 fatty acid supplements (1800 mg/day) and 20 were placebo (olive oil) supplements (600 mg/day) for 180 days.

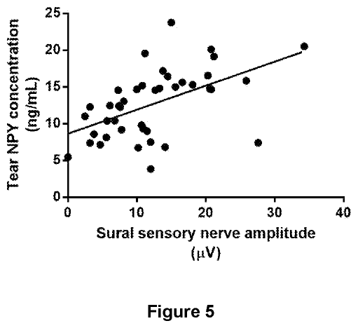

The primary outcome of this trial was that, relative to placebo, oral omega-3 supplementation for 180 days led to an improvement in the density of corneal nerves (i.e, there was a neuroregenerative effect observed in the corneal nerves) (FIG. 5; measured using the CNFL parameter with IVCM) (Britten-Jones et al., Investigating the neuroprotective effect of Oral Omega-3 Fatty acid Supplementation in type 1 diabetes (nPROOFS1): a randomised, placebo-controlled trial, Diabetes (2021) 70:1794-1806.

An analysis of the relative change in tear NPY levels in the same study population was performed using analysis of covariance (ANCOVA).

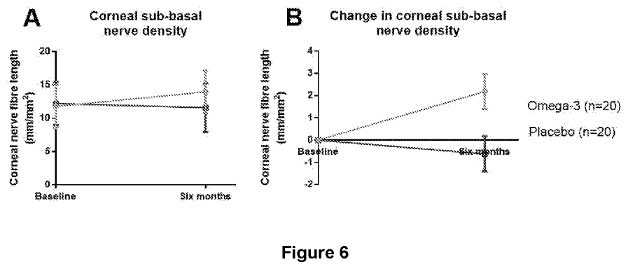

At day 180, the change in tear NPY levels in the omega-3 treated group relative to placebo was 2.38 ng/mL (95% confidence interval: 0.94 to 3.82 ng/mL; FIG. 6). The inter-group difference was statistically significant (p=0.002).

### Change in Corneal Nerve Structural Parameters Vs Baseline Tear NPY

Of the participants with type 1 diabetes who were treated with oral omega-3 fatty acids for six months, those who had a tear NPY level of 12 ng/mL or more at baseline (n=12) all showed an improvement in CNFL at the study endpoint, suggesting that they were more likely to be responders to an anti-inflammatory and/or neuroprotective intervention (in this case the representative intervention being an oral omega-3 supplement). Participants with tear NPY level of about 12 ng/ml or more also had a higher likelihood of achieving a clinically significant improvement (defined as >2 mm/mm² in CNFL) with the intervention, compared to those with tear NPY levels≤12 ng/mL (75% with clinically significant improvement in the group with tear NPY>12 ng/mL vs 50% in group with tear NPY≤12 ng/mL).

The claims defining the invention are as follows:

1. A method of diagnosing the presence of or pre-disposition for corneal nerve damage or loss in a subject, said method comprising the steps of:
a) determining the level of neuropeptide Y (NPY) in a tear fluid sample from the subject; and
b) comparing the level of the NPY to a reference level of the NPY,
wherein a decreased level of the NPY compared to the reference level is indicative of the presence of or pre-disposition for corneal nerve damage or loss.

2. The method of claim 1, wherein the decreased level of the NPY indicates reduced corneal nerve fibre length (CNFL) and/or corneal nerve fibre density (CNFD) in the subject.

3. A method of diagnosing the presence of or pre-disposition for corneal neuropathy, corneal neuropathic pain, or corneal neuralgia, said method comprising the steps of:
a) determining the level of neuropeptide Y (NPY) in a tear fluid sample from the subject; and
b) comparing the level of the NPY to a reference level of the NPY,
wherein a decreased level of the NPY compared to the reference level indicates that indicates that said subject is suffering from or is at risk of corneal neuropathy, corneal neuropathic pain, or corneal neuralgia.

4. The method of claim 3, wherein the decreased level of the NPY indicates increased corneal nerve damage in the subject.

5. The method of claim 4, wherein the decreased level of the NPY indicates reduced corneal nerve fibre length (CNFL) and/or corneal nerve fibre density (CNFD) in the subject.

6. A method of diagnosing the presence of or pre-disposition for an ocular disease, said method comprising the steps of:
a) determining the level of neuropeptide Y (NPY) in a tear fluid sample from the subject; and
b) comparing the level of the NPY to a reference level of the NPY,
wherein a decreased level of the NPY compared to the reference level indicates that indicates that said subject is suffering from or is at risk of developing an ocular disease.

7. The method of claim 6, wherein the decreased level of the NPY indicates increased corneal nerve damage or loss in the subject.

8. The method of claim 6 or claim 7, wherein the decreased level of the NPY indicates reduced corneal nerve fibre length (CNFL) and/or corneal nerve fibre density (CNFD) in the subject.

9. A method of diagnosing the presence of or pre-disposition for peripheral neuropathy, said method comprising the steps of:
a) determining the level of neuropeptide Y (NPY) in a tear fluid sample from the subject; and
b) comparing the level of the NPY to a reference level of the NPY,
wherein a decreased level of the NPY compared to the reference level indicates that indicates that said subject is suffering from or is at risk of developing peripheral neuropathy.

10. The method of claim 9, wherein the decreased level of the NPY indicates increased corneal nerve damage or loss in the subject.

11. The method of claim 9 or claim 10, wherein the peripheral neuropathy is diabetic peripheral neuropathy.

12. The method of any one of claims 9 to 11, wherein the decreased level of the NPY indicates reduced corneal nerve fibre length (CNFL) and/or corneal nerve fibre density (CNFD) in the subject.

13. The method of any one of claims 9 to 12, wherein the decreased level of the NPY indicates reduced sural sensory nerve amplitude.

14. A method of evaluating the level of corneal nerve damage or loss in a subject, said method comprising the steps of:
a) determining the level of neuropeptide Y (NPY) in a tear fluid sample from the subject
b) correlating the level of the NPY with severity of corneal nerve damage or loss.

15. A method of evaluating the level of corneal neuropathy, corneal neuropathic pain, or corneal neuralgia, said method comprising the steps of:
a) determining the level of neuropeptide Y (NPY) in a tear fluid sample from the subject; and
b) correlating the level of the NPY with severity of corneal neuropathy, corneal neuropathic pain, or corneal neuralgia.

16. The method of claim 15, wherein the level of the NPY indicates the level of corneal nerve damage or loss, wherein a decreased level of NPY indicates increased corneal nerve damage or loss.

17. A method of evaluating the level of peripheral neuropathy, said method comprising the steps of:
a) determining the level of neuropeptide Y (NPY) in a tear fluid sample from the subject; and
b) correlating the level of the NPY with severity of peripheral neuropathy.

18. The method of claim 17, wherein the level of the NPY indicates the level of corneal nerve damage or loss, wherein a decreased level of NPY indicates increased corneal nerve damage or loss.

19. The method of claim 17 or claim 18, wherein the peripheral neuropathy is diabetic peripheral neuropathy.

20. The method of any one of claims 17 to 19, wherein the method is for risk stratification or treatment stratification.

21. A method of prognosis of a subject having corneal nerve damage or loss, the method comprising:
a) determining the level of neuropeptide Y (NPY) in a tear fluid sample from the subject; and
b) correlating said level of NPY to likely outcome of said corneal nerve damage or loss.

22. A method of prognosis of a subject having corneal neuropathy, corneal neuropathic pain, or corneal neuralgia, the method comprising:
a) determining the level of neuropeptide Y (NPY) in a tear fluid sample from the subject; and
b) correlating said level of NPY to likely outcome of said corneal neuropathy, corneal neuropathic pain, or corneal neuralgia.

23. The method of claim 22, wherein the level of the NPY correlates with the level of corneal nerve damage or loss, wherein a decreased level of NPY indicates increased corneal nerve damage or loss.

24. A method of prognosis of a subject having peripheral neuropathy, the method comprising:
a) determining the level of neuropeptide Y (NPY) in a tear fluid sample from the subject; and
b) correlating said level of NPY to likely outcome of said peripheral neuropathy.

25. The method of claim 24, wherein the level of the NPY correlates with the level of corneal nerve damage or loss, wherein a decreased level of NPY indicates increased corneal nerve damage or loss.

26. The method of claim 24 or claim 25, wherein the peripheral neuropathy is diabetic peripheral neuropathy.

27. The method of any one of claims 21 to 26, wherein the method is for monitoring treatment efficacy or disease progression.

28. A method of monitoring treatment efficacy of a subject having corneal nerve damage or loss, corneal neuropathy, corneal neuropathic pain, or corneal neuralgia, ocular disease, or peripheral neuropathy, the method comprising:
a) determining the level of neuropeptide Y (NPY) in a tear fluid sample from the subject; and
b) correlating said level of NPY to a reference level indicative of a responder or non-responder to the treatment, or comparing said level to a baseline level of NPY before initiation of treatment; and
c) optionally, continuing treatment when the subject is determined to be a responder or has an increased level of NPY compared to the baseline level; or switching the treatment when the subject is determined to be a non-responder, wherein switching the treatment comprises increasing a dose of the treatment administered, adding one or more of agents to the treatment regimen, discontinuing the treatment and initiating a different treatment regimen.

29. The method of claim 28, wherein the peripheral neuropathy is diabetic peripheral neuropathy.

30. The method of any one of claims 1 to 29, wherein the NPY comprises an amino acid sequence shown in SEQ ID NO: 2, or a fragment thereof.

31. The method of any one of claims 1 to 13, or claim 30, wherein the reference level of the NPY in a sample from a normal subject.

32. The method of any one of claims 1 to 13, or claim 30 or 31, wherein the reference level of the NPY is a threshold level.

33. The method of claim 32, wherein the threshold level is a cut-off value.

34. The method of any one of claims 1 to 33, wherein the method further comprises obtaining the tear fluid sample.

35. The method of any one of claims 1 to 34, further comprising a psychological, behavioural, physiological and/or genetic assessment of the subject.

36. The method of any one of claims 1 to 35, further comprising selecting a treatment or modifying a treatment, based on the diagnosis, evaluation, or prognosis.

37. The method of any one of claims 1 to 36, further comprising administering to the subject a therapeutically effective amount of a therapeutic.

38. Use of ocular tear fluid neuropeptide Y (NPY) as a biomarker for corneal nerve damage or loss, corneal neuropathy, corneal neuropathic pain, or corneal neuralgia, ocular disease, or peripheral neuropathy.
